(19) United States
(12) Patent Application Publication
(10) Pub. No.: US 2025/0136976 A1
(43) Pub. Date: May 1, 2025

(54) USE OF ETV3 OR ETV6 INHIBITORS FOR BLOCKING THE DIFFERENTIATION OF MONOCYTES INTO DENDRITIC CELLS

(71) Applicants: INSERM (INSTITUT NATIONAL DE LA SANTÉ ET DE LA RECHERCHE MÉDICALE), Paris (FR); INSTITUT CURIE, Villejuif Cedex (FR)

(72) Inventors: Elodie SEGURA, Paris Cedex 05 (FR); Javiera VILLAR, Paris (FR)

(21) Appl. No.: 18/684,020

(22) PCT Filed: Aug. 31, 2022
(86) PCT No.: PCT/EP2022/074147

§ 371 Date: Feb. 15, 2024

(30) Foreign Application Priority Data
Sep. 1, 2021 (EP) 21306195.5

(51) Int. Cl. C12N 15/113; A61K 31/713; A61P 37/02

(52) U.S. Cl. CPC C12N 15/113; A61K 31/713; A61P 37/02; C12N 2310/14; C12N 2310/531

(57) ABSTRACT

In inflamed tissues, monocytes differentiate into macrophages (mo-Mac) or dendritic cells (mo-DC). In chronic non-resolving inflammation, mo-DC are major drivers of pathogenic events. Manipulating monocyte differentiation would therefore represent an attractive therapeutic strategy. Here the inventors show that the transcriptional repressors ETV3 and ETV6 control monocyte differentiation into mo-DC. To validate the physiological relevance of these findings, the inventors generated mice deficient for ETV6 in monocytes. Deficient mice show spontaneous expression of interferon-stimulated genes, confirming that ETV6 regulates interferon responses in vivo. Furthermore, deficient mice display impaired mo-DC differentiation during peritonitis and less severe symptoms in experimental autoimmune encephalomyelitis. The findings identify ETV3 and ETV6 as a therapeutic target to redirect monocyte differentiation in inflammatory disorders.

### FIELD OF THE INVENTION

The present invention is in the field of medicine, in particular immunology.

### BACKGROUND OF THE INVENTION

Monocytes and monocyte-derived cells are central players in the initiation and resolution of inflammatory responses. In chronic inflammatory diseases, monocyte-derived antigen-presenting cells become major drivers of the physiopathology by stimulating pathogenic T cells. Blocking monocyte differentiation therefore represents an attractive therapeutic strategy. A major hurdle is the paucity of molecular targets, due to a limited knowledge of the molecular regulation of monocyte fate commitment.

Circulating monocytes infiltrate mucosal or inflamed tissues where they differentiate into macrophages (mo-Mac) or dendritic cells (mo-DC) (Coillard and Segura, 2019; Guilliams et al., 2018; Jakubzick et al., 2017). Mo-Mac are generally involved in homeostasis and tissue repair, while mo-DC present antigens to T cells directly in tissues. However, in chronic non-resolving inflammation, T cell stimulation by mo-DC becomes deleterious. In Crohn's disease, rheumatoid arthritis and psoriasis, mo-DC secrete high amounts of IL-23 and stimulate Th17 cells, two major drivers of the physiopathology (Evans et al., 2009; Kamada et al., 2008; Segura et al., 2013; Zaba et al., 2009). In mouse models, mo-DC induce pathogenic T cells that mediate tissue damage in experimental autoimmune encephalomyelitis (EAE) (Croxford et al., 2015) and colitis (Arnold et al., 2016; Zigmond et al., 2012). Blocking monocyte differentiation has therefore emerged as a potential therapeutic strategy for inflammatory disorders. Pharmacological inhibition of monocyte recruitment suppresses the development of colitis (Bhatia et al., 2008) and the severity of EAE (Ge et al., 2012). Inducing monocyte apoptosis with nanoparticles reduces inflammation and disease symptoms in colitis, EAE, peritonitis and virus-induced encephalitis (Getts et al., 2014). Finally, impairing monocyte survival and differentiation via M-CSF receptor blockade reduces inflammation in arthritis (Garcia et al., 2016; Toh et al., 2014). However, a major caveat of these approaches is the potential adverse effects due to the disruption of homeostatic events, such as the differentiation of mo-Mac involved in resolution of inflammation. Such deleterious effects have been reported for cardiac repair (Leblond et al., 2015) and skeletal muscle regeneration (Segawa et al., 2008). Manipulating monocyte fate commitment towards mo-DC versus mo-Mac would therefore provide an attractive alternative strategy. This would require a better understanding of the molecular regulators orchestrating monocyte fate decision.

Monocyte fate is not transcriptionally imprinted (Goudot et al., 2017; Mildner et al., 2017). Instead, monocytes respond to micro-environmental cues that can redirect their fate. Using in vitro models of human monocyte differentiation, we and others have shown that IL-4 signaling is essential to induce mo-DC differentiation (Goudot et al., 2017; Sander et al., 2017). Transcription factors involved in this process include IRF4, aryl hydrocarbon receptor, BLIMP-1 and the nuclear receptor corepressor 2 (NCOR2) (Goudot et al., 2017; Sander et al., 2017). What controls the balance of monocyte differentiation into mo-Mac versus mo-DC remains unclear.

### SUMMARY OF THE INVENTION

The present invention is defined by the claims. In particular, the present invention relates to use of ETV3 or ETV6 inhibitors for blocking the differentiation of monocytes into dendritic cells.

### DETAILED DESCRIPTION OF THE INVENTION

In inflamed tissues, monocytes differentiate into macrophages (mo-Mac) or dendritic cells (mo-DC). In chronic non-resolving inflammation, mo-DC are major drivers of pathogenic events. Manipulating monocyte differentiation would therefore represent an attractive therapeutic strategy. However, what regulates the balance of mo-DC versus mo-Mac fate commitment remains unclear. Here the inventors show that the transcriptional repressors ETV3 and ETV6 control monocyte differentiation into mo-DC. Mechanistically, the inventors find that ETV3 and ETV6 repress mo-Mac development and inhibit the expression of interferon-stimulated genes. Moreover, they demonstrate that activation of the type I interferon pathway promotes mo-Mac differentiation. To validate the physiological relevance of these findings, the inventors generated mice deficient for ETV6 in monocytes. Deficient mice show spontaneous expression of interferon-stimulated genes, confirming that ETV6 regulates interferon responses in vivo. Furthermore, deficient mice display impaired mo-DC differentiation during peritonitis and less severe symptoms in experimental autoimmune encephalomyelitis. The findings allow a better understanding of the molecular control of monocyte fate decision and identify ETV3 and ETV6 as a therapeutic target to redirect monocyte differentiation in inflammatory disorders.

Accordingly, the first object of the present invention relates to a method for blocking differentiation of monocytes into dendritic cells in a subject in need thereof comprising administering to the subject a therapeutic effective amount of a ETV6 or ETV3 inhibitor.

As used herein the term “monocyte” has its general meaning in the art and is a large mononuclear phagocyte of the peripheral blood. Monocytes vary considerably, ranging in size from 10 to 30 μm in diameter. The nucleus to cytoplasm ratio ranges from 2:1 to 1:1. The nucleus is often band shaped (horseshoe), or reniform (kindey-shaped). It may fold over on top of itself, thus showing brainlike convolutions. No nucleoli are visible. The chromatin pattern is fine, and arranged in skein-like strands. The cytoplasm is abundant and appears blue gray with many fine azurophilic granules, giving a ground glass appearance in Giemsa staining. Vacuoles may be present. More preferably, the expression of specific surface antigens is used to determine whether a cell is a monocyte cell. The main phenotypic markers of human monocyte cells include CD11b, CD11c, CD33 and CD115. Generally, human monocyte cells express CD9, CD11b, CD11c, CDw12, CD13, CD15, CDw17, CD31, CD32, CD33, CD35, CD36, CD38, CD43, CD49b, CD49e, CD49f, CD63, CD64, CD65s, CD68, CD84, CD85, CD86, CD87, CD89, CD91, CDw92, CD93, CD98, CD101, CD102, CD111, CD112, CD115, CD116, CD119, CDw121b, CDw123, CD127, CDw128, CDw131, CD147, CD155, CD156a, CD157, CD162, CD163, CD164, CD168, CD171, CD172a, CD180, CD206, CD131a1, CD213a2, CDw210, CD226, CD281, CD282, CD284, CD286 and optionally CD4, CD14, CD16, CD40, CD45RO, CD45RA, CD45RB, CD62L, CD74, CD142 and CD170, CD181, CD182, CD184, CD191, CD192, CD194, CD195, CD197, CX3CR1.

As used herein, the term “dendritic cell” or “DC” refers to a sub-type of antigen presenting cells that are characterized at the morphological level by numerous membrane processes that extend out from the main cell body (similar to dendrites on neurons) and at the biochemical level by cell surface expression of MHC class II molecules and lack of expression of one or more of CD3, CD14, CD19, CD56 and/or CD66b. Subsets of dendritic cells express on their cell surface CD11a, CD11c, CD50, CD54, CD58, CD102, CD80 and/or CD86. Some DCs also express toll-like receptors 2, 3, 4, 7 and/or 9. The term “mo-dendritic cell” or “mo-DC” refers to dendric cells that result from the differentiation of monocyte.

In some embodiments, the subject suffer from an inflammatory disease. Thus the ETV3 or ETV6 inhibitor is particularly suitable for the treatment of inflammatory diseases.

As used herein, the term “inflammatory disease” has its general meaning in the art and refers to a condition in a patient characterized by inflammation, preferably chronic inflammation. Moreover, inflammation may or may not be caused by an autoimmune disorder. Thus, certain inflammatory diseases may be characterized as both autoimmune and inflammatory diseases.

In some embodiments, the patient suffers from an inflammatory diseases selected from the group consisting of arthritis, rheumatoid arthritis, acute arthritis, chronic rheumatoid arthritis, gouty arthritis, acute gouty arthritis, chronic inflammatory arthritis, degenerative arthritis, infectious arthritis, Lyme arthritis, proliferative arthritis, psoriatic arthritis, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, hidradenitis suppurativa, dermatitis including contact dermatitis, chronic contact dermatitis, allergic dermatitis, allergic contact dermatitis, dermatitis herpetiformis, and atopic dermatitis, x-linked hyper IgM syndrome, urticaria such as chronic allergic urticaria and chronic idiopathic urticaria, including chronic autoimmune urticaria, polymyositis/dermatomyositis, juvenile dermatomyositis, toxic epidermal necrolysis, scleroderma, systemic scleroderma, sclerosis, systemic sclerosis, multiple sclerosis (MS), spino-optical MS, primary progressive MS (PPMS), relapsing remitting MS (RRMS), progressive systemic sclerosis, atherosclerosis, arteriosclerosis, sclerosis disseminata, and ataxic sclerosis, peritonitis, inflammatory bowel disease (IBD), Crohn's disease, colitis, ulcerative colitis, colitis ulcerosa, microscopic colitis, collagenous colitis, colitis polyposa, necrotizing enterocolitis, transmural colitis, autoimmune inflammatory bowel disease, pyoderma gangrenosum, erythema nodosum, primary sclerosing cholangitis, episcleritis, respiratory distress syndrome, adult or acute respiratory distress syndrome (ARDS), meningitis, inflammation of all or part of the uvea, iritis, choroiditis, an autoimmune hematological disorder, rheumatoid spondylitis, sudden hearing loss, IgE-mediated diseases such as anaphylaxis and allergic and atopic rhinitis, encephalitis, Rasmussen's encephalitis, limbic and/or brainstem encephalitis, uveitis, anterior uveitis, acute anterior uveitis, granulomatous uveitis, nongranulomatous uveitis, phacoantigenic uveitis, posterior uveitis, autoimmune uveitis, glomerulonephritis (GN), idiopathic membranous GN or idiopathic membranous nephropathy, membrano- or membranous proliferative GN (MPGN), rapidly progressive GN, allergic conditions, autoimmune myocarditis, leukocyte adhesion deficiency, systemic lupus erythematosus (SLE) or systemic lupus erythematodes such as cutaneous SLE, subacute cutaneous lupus erythematosus, neonatal lupus syndrome (NLE), lupus erythematosus disseminatus, lupus (including nephritis, cerebritis, pediatric, non-renal, extra-renal, discoid, alopecia), juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, immune responses associated with acute and delayed hypersensitivity mediated by cytokines and T-lymphocytes, tuberculosis, sarcoidosis, granulomatosis, lymphomatoid granulomatosis, Wegener's granulomatosis, agranulocytosis, vasculitides, including vasculitis, large vessel vasculitis, polymyalgia rheumatica, giant cell (Takayasu's) arteritis, medium vessel vasculitis, Kawasaki's disease, polyarteritis nodosa, microscopic polyarteritis, CNS vasculitis, necrotizing, cutaneous, hypersensitivity vasculitis, systemic necrotizing vasculitis, and ANCA-associated vasculitis, such as Churg-Strauss vasculitis or syndrome (CSS), temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), pernicious anemia (anemia pemiciosa), Addison's disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Bechet's or Behcet's disease, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus, optionally pemphigus vulgaris, pemphigus foliaceus, pemphigus mucus-membrane pemphigoid, pemphigus erythematosus, autoimmune polyendocrinopathies, Reiter's disease or syndrome, immune complex nephritis, antibody-mediated nephritis, neuromyelitis optica, polyneuropathies, chronic neuropathy, IgM polyneuropathies, IgM-mediated neuropathy, thrombocytopenia, thrombotic thrombocytopenic purpura (TTP), idiopathic thrombocytopenic purpura (ITP), autoimmune orchitis and oophoritis, primary hypothyroidism, hypoparathyroidism, autoimmune thyroiditis, Hashimoto's disease, chronic thyroiditis (Hashimoto's thyroiditis); subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis, allergic encephalomyelitis, experimental allergic encephalomyelitis (EAE), myasthenia gravis, thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, giant cell hepatitis, chronic active hepatitis or autoimmune chronic active hepatitis, lymphoid interstitial pneumonitis, bronchiolitis obliterans (non-transplant) vs NSIP, Guillain-Barre syndrome, Berger's disease (IgA nephropathy), idiopathic IgA nephropathy, linear IgA dermatosis, primary biliary cirrhosis, pneumonocirrhosis, autoimmune enteropathy syndrome, Celiac disease, Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AGED), autoimmune hearing loss, opsoclonus myoclonus syndrome (OMS), polychondritis such as refractory or relapsed polychondritis, pulmonary alveolar proteinosis, amyloidosis, scleritis, a non-cancerous lymphocytosis, a primary lymphocytosis, which includes monoclonal B cell lymphocytosis, optionally benign monoclonal gammopathy or monoclonal gammopathy of undetermined significance, MGUS, peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases, diabetic nephropathy, Dressler's syndrome, alopecia greata, CREST syndrome (calcinosis, Raynaud's phenomenon, esophageal dysmotility, sclerodactyl), and telangiectasia), male and female autoimmune infertility, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis, or Fuch's cyclitis, Henoch-Schonlein purpura, human immunodeficiency virus (HIV) infection, echovirus infection, cardiomyopathy, Alzheimer's disease, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant cell polymyalgia, endocrine ophthamopathy, chronic hypersensitivity pneumonitis, keratoconjunctivitis sicca, epidemic keratoconjunctivitis, idiopathic nephritic syndrome, minimal change nephropathy, benign familial and ischemia-reperfusion injury, retinal autoimmunity, joint inflammation, bronchitis, chronic obstructive airway disease, silicosis, aphthae, aphthous stomatitis, arteriosclerotic disorders, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired splenic atrophy, infertility due to antispermatozoan antibodies, non-malignant thymoma, vitiligo, SCID and Epstein-Barr virus-associated diseases, acquired immune deficiency syndrome (AIDS), parasitic diseases such as Lesihmania, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, peripheral neuropathy, autoimmune polyglandular syndrome type I, adult-onset idiopathic hypoparathyroidism (AOIH), alopecia totalis, dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia, autoimmune disorders associated with collagen disease, rheumatism, neurological disease, ischemic re-perfusion disorder, reduction in blood pressure response, vascular dysfunction, antgiectasis, tissue injury, cardiovascular ischemia, hyperalgesia, cerebral ischemia, and disease accompanying vascularization, allergic hypersensitivity disorders, glomerulonephritides, reperfusion injury, reperfusion injury of myocardial or other tissues, dermatoses with acute inflammatory components, acute purulent meningitis or other central nervous system inflammatory disorders, ocular and orbital inflammatory disorders, granulocyte transfusion-associated syndromes, cytokine-induced toxicity, acute serious inflammation, chronic intractable inflammation, pyelitis, pneumonocirrhosis, diabetic retinopathy, diabetic large-artery disorder, endarterial hyperplasia, peptic ulcer, valvulitis, and endometriosis.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “ETV3” has its general meaning in the art and refers to the ETS translocation variant 3. The term is also known as Mitogenic Ets transcriptional suppressor, METS, PE1 or PE-1. An exemplary amino acid sequence for ETV3 is shown as SEQ ID NO:1.

>sp|P41162|ETV3_HUMAN ETS translocation variant 3
OS = Homo sapiens OX = 9606 GN = ETV3 PE = 1 SV =
2
SEQ ID NO: 1
MKAGCSIVEKPEGGGGYQFPDWAYKTESSPGSRQIQLWHFILELLQKEEF
RHVIAWQQGEYGEFVIKDPDEVARLWGRRKCKPQMNYDKLSRALRYYYNK
RILHKTKGKRFTYKFNFNKLVMPNYPFINIRSSGVVPQSAPPVPTASSRF
HFPPLDTHSPTNDVQPGRFSASSLTASGQESSNGTDRKTELSELEDGSAA
DWRRGVDPVSSRNAIGGGGIGHQKRKPDIMLPLFARPGMYPDPHSPFAVS
PIPGRGGVLNVPISPALSLTPTIFSYSPSPGLSPFTSSSCFSFNPEEMKH
YLHSQACSVFNYHLSPRTFPRYPGLMVPPLQCQMHPEESTQFSIKLQPPP
VGRKNRERVESSEESAPVTTPTMASIPPRIKVEPASEKDPESLRQSAREK
EEHTQEEGTVPSRTIEEEKGTIFARPAAPPIWPSVPISTPSGEPLEVTED
SEDRPGKEPSAPEKKEDALMPPKLRLKRRWNDDPEARELSKSGKFLWNGS
GPQGLATAAADA

As used herein, the term “ETV6” has its general meaning in the art and refers to the Transcription factor ETV6. The term is also known as ETS translocation variant 6, ETS-related protein Tell, TEL or TEL 1. An exemplary amino acid sequence for ETV6 is shown as SEQ ID NO:2.

>sp|P41212|ETV6_HUMAN Transcription factor ETV6
OS = Homo sapiens OX = 9606 GN = ETV6 PE = 1
SV = 1
SEQ ID NO: 2
MSETPAQCSIKQERISYTPPESPVPSYASSTPLHVPVPRALRMEEDSIRL
PAHLRLQPIYWSRDDVAQWLKWAENEFSLRPIDSNTFEMNGKALLLLTKE
DFRYRSPHSGDVLYELLQHILKQRKPRILFSPFFHPGNSIHTQPEVILHQ
NHEEDNCVQRTPRPSVDNVHHNPPTIELLHRSRSPITTNHRPSPDPEQRP
LRSPLDNMIRRLSPAERAQGPRPHQENNHQESYPLSVSPMENNHCPASSE
SHPKPSSPRQESTRVIQLMPSPIMHPLILNPRHSVDFKQSRLSEDGLHRE
GKPINLSHREDLAYMNHIMVSVSPPEEHAMPIGRIADCRLLWDYVYQLLS
DSRYENFIRWEDKESKIFRIVDPNGLARLWGNHKNRTNMTYEKMSRALRH
YYKLNIIRKEPGQRLLFRFMKTPDEIMSGRTDRLEHLESQELDEQIYQED
EC

As used herein, the term “ETV3 inhibitor” and “ETV6 inhibitor” refer to refers to a compound natural or not which is capable of inhibiting the activity or expression of ETV3 and ETV6 respectively. The term encompasses any ETV3 or ETV6 inhibitor that is currently known in the art or that will be identified in the future, and includes any chemical entity that, upon administration to a patient, results in inhibition or down-regulation of a biological activity associated with activation of the ETV3 or ETV6. The term also encompasses inhibitor of expression.

In some embodiments, the ETV3 or ETV6 inhibitor is a small organic molecule.

In some embodiments, the ETV3 or ETV6 inhibitor is an inhibitor of ETV3 or ETV6 expression.

An “inhibitor of expression” refers to a natural or synthetic compound that has a biological effect to inhibit the expression of a gene.

In some embodiments, said inhibitor of gene expression is a siRNA, an antisense oligonucleotide or a ribozyme. For example, anti-sense oligonucleotides, including anti-sense RNA molecules and anti-sense DNA molecules, would act to directly block the translation of ETV3 or ETV6 mRNA by binding thereto and thus preventing protein translation or increasing mRNA degradation, thus decreasing the level of ETV3 or ETV6, and thus activity, in a cell.

For example, antisense oligonucleotides of at least about 15 bases and complementary to unique regions of the mRNA transcript sequence encoding ETV3 or ETV6 can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of expression for use in the present invention. ETV3 or ETV6 gene expression can be reduced by contacting a subject or cell with a small double stranded RNA (dsRNA), or a vector or construct causing the production of a small double stranded RNA, such that ETV3 or ETV6 gene expression is specifically inhibited (i.e. RNA interference or RNAi). Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically cells expressing ETV3 or ETV6. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

As used herein, the term “therapeutically effective amount” is meant a sufficient amount of the drug (i.e. ETV3 or ETV6 inhibitor) for treating or reducing the symptoms at reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination with the active ingredients; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Typically the active ingredient of the present invention (i.e. ETV3 or ETV6 inhibitor) is combined with pharmaceutically acceptable excipients, and optionally sustained-release matrices, such as biodegradable polymers, to form pharmaceutical compositions. The term “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. In the pharmaceutical compositions of the present invention, the active ingredients of the invention can be administered in a unit administration form, as a mixture with conventional pharmaceutical supports. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

The invention will be further illustrated by the following figures and examples. However, these examples and figures should not be interpreted in any way as limiting the scope of the present invention.

### FIGURES

FIG. 1: ETV3 and ETV6 are essential for mo-DC differentiation. (A) Human Monocytes were cultured with M-CSF, IL-4 and TNFα. ETV3 or ETV6 expression was silenced using a lentivirus containing shRNA. Protein quantification by Western Blot after 5 days. Actin was used as loading control. Representative results are shown (n=8). (B) Mo-mac and mo-DC differentiation from monocytes after 5 days was assessed by flow cytometry. One representative donor is shown. Median is shown (n=8 in 3 independent experiments). Paired-one way Anova. For all panels: *P<0.05, **P<0.01, ***P<0.001, ****P<0.0001.

FIG. 2: Etv6 controls mo-DC differentiation during in vivo inflammation in mouse (A) Experimental set up of peritonitis model. (B) CD226 and ICAM-2 expression in CD11b+CD115+ cells from peritoneal lavage of Etv6fl/fl (WT) and Cx3cr1-Etv6Δ (KO) mice. Results from one representative pair of littermates are shown for each setting. (C) Numbers of monocytes, mo-DC, mo-Mac or resident macrophages (Res Mac) in the peritoneal lavage. Each symbol represents one mouse. Median is shown (n=12 in 3 independent experiments). Unpaired t-test between WT and KO. Two-way Anova with a Tukey post-test between steady state and inflammation groups. (D) Experimental autoimmune encephalomyelitis (EAE) was induced by injection of MOG peptide. Experimental set up. (E) Mean clinical score is shown. Bars represent SEM (n=16 for WT and 18 for KO in 4 independent experiments). Multiple Mann-Whitney tests between WT and KO groups. (F) Peak clinical score. Median is shown. Each dot represents one mouse (median of n=16 for WT and n=18 for KO in 4 independent experiments). Mann-Whitney test. For all panels: *P<0.05, **P<0.01, ***P<0.001, ****P<0.0001.

### EXAMPLE

### Methods

### Human Samples

Buffy coats from healthy donors (both male and female donors) were obtained from Etablissement Frangais du Sang (Paris) in accordance with INSERM ethical guidelines. According to French Public Health Law (art L 1121-1-1, art L 1121-1-2), written consent and IRB approval are not required for human non-interventional studies.

### Mouse Strains

Cx3Cr1-CreER were obtained from Jackson Laboratories (Stock #021160). Cx3Cr1-CreER express the enhanced yellow fluorescent protein (EYFP) from endogenous Cx3cr1 promoter/enhancer elements. Etv6^{flox/flox} mice were obtained from H.Hock (Hock et al., 2004). Cx3cr1-Etv6^{Δ} were generated by crossing Cx3Cr1-CreER^{+/-} mice with Etv6^{flox/flox} mice. For Cx3cr1-Etv6^{Δ} mice, Cx3Cr1-CreER^{-/-} Etv6^{flox/flox} littermates were used as WT controls. CD11c-Etv6^{Δ} have been previously described (Lau et al., 2018). All mice were on C57BL/6 background. Mice were maintained under specific pathogen-free conditions at the animal facility of Institut Curie in accordance with institutional guidelines. Both male and female mice were used and sacrificed at age 7-9 weeks. All animal procedures were in accordance with the guidelines and regulations of the French Veterinary Department and approved by the local ethics committee.

### Monocyte Isolation and Culture

Peripheral Blood Mononuclear Cells (PBMC) were prepared by centrifugation on a Ficoll gradient (Lymphoprep, StemCell). Blood CD14+ monocytes were isolated from healthy donors' PBMC by positive selection using magnetic beads (Miltenyi). Monocytes were 95%-98% CD14⁺CD16⁻ as assessed by flow cytometry. Monocytes (2×10⁶ cells/mL) were cultured for 5 days in RPMI-Glutamax medium (GIBCO) supplemented with antibiotics (penicillin and streptomicin) and 10% Fetal Calf Serum in the presence or absence of 100 ng/mL M-CSF (Miltenyi), 5 ng/mL IL-4 (Miltenyi) and 5 ng/mL TNF-α (R&D Biotechne). Cytokines were added only at the start of the culture, and medium was not refreshed during the course of the culture. CD16+ or CD1a+ cell populations were isolated by cell sorting on a FACSAria instrument (BD Biosciences).

### Flow Cytometry of Human Cells

Human cells were stained in PBS containing 0.5% human AB serum and 2 mM EDTA with APC anti-CD1a (Biolegend, clone HI149), FITC anti-CD16 (Biolegend, clone 3G8), PE-Cy7 anti-CD163 (Biolegend, clone GHI/61), PE anti-CD1b (eBioscience, clone eBioSN13). DAPI (Fischer Scientific, 100 ng/mL) was added immediately prior to acquisition on a FacsVerse instrument (BD Biosciences) or MACSQuant (Miltenyi) instrument. Data was analyzed with FlowJo (FlowJo LLC).

### Flow Cytometry of Mouse Tissues

Cells were stained in PBS containing BSA 0.5% and 2 mM EDTA for 30-45 min on ice. Antibodies used were anti-CD115 BUV 395 (BD Bioscience, clone AFS98), anti-TCRβ BUV737 (BD Bioscience, clone H57-597), anti-CD19 BV480 (BD Bioscience clone 1D3), anti-TCRb BV480 (BD Bioscience, clone H57-597), anti-NK1.1 BV480 (BD Bioscience, clone PK136), anti-SiglecF BV480 (BD Bioscience, clone E50-2440), anti-Ly6G BV605 (Biolegend, clone 1A8), anti-MHC II BV650 (Biolegend, clone M5/i 14.15.2), anti-CCR2 BV711 (BD Bioscience, clone 475301), anti-CD11c BV785 (Biolegend, clone N418), anti-CD226 PE (Biolegend, clone 10E5), anti-CD11b PE da594 (BD Bioscience, clone M1/70), anti-CD11b PerCPCy5.5 (BD Biosciences, clone M1/70), anti-CD16/32 PECy7 (Biolegend, clone 93), anti-TIM4 APC (Biolegend, clone RMT4-54), anti-Ly6C Alexa 700 (Biolegend, clone HK1.4), and anti-ICAM2 Biotin (Biolegend, clone 3C4) followed by Streptavidin BV421 (Invitrogen). After washing, cells were resuspended in staining buffer containing DAPI (Fischer Scientific, 100 ng/mL). Cells were acquired on a ZE5 flow cytometer (Bio-Rad). Supervised analysis was performed using FlowJo software.

shRNA Interference

shRNA (all from Sigma) against ETV3 (sh1: NM_005240-TRCN0000013930, sh2: NM_005240—TRCN0000013931, sh3: NM_005240-TRCN0000013932), ETV6 (sh1: NM_001987—TRCN0000003853, sh2: NM_001987-TRCN0000003854, sh3: NM_001987-TRCN0000003855), or nontargeting control shRNA (MISSION shRNA SHC002) were in the LKO.1-puro vector (MISSION® Sigma). Viral particles were produced by transfection of 293FT cells in 6-well plates with 3 mg DNA and 8 uL TransIT-293 (Mirus Bio) per well: for VSV-G pseudotyped SIVmac VLPs, 0.4 mg CMV-VSVG and 2.6 mg pSIV3+; for shRNA vectors, 0.4 mg CMV-VSV-G, 1 mg psPAX2 and 1.6 mg LKO1puro-derived shRNA vector. One day after 293FT cells transfection, medium was replaced by fresh culture medium. Viral supernatants were harvested 1 day later and filtered through 0.45 μm filters. Freshly isolated CD14+ monocytes were infected with viral particles containing the control vector or individual shRNA vectors, and cultured as above. Puromycin (InvivoGen) was added 2 days later (2 mg/mL). At day 5, cells were harvested for analysis.

### Immuno Blot

Cells were lysed in RIPA buffer (Thermo Scientific) supplemented with complete Mini EDTA-free protease inhibitor cocktail (Roche), at 1×10⁶ cells in 100 μL of lysis buffer. Post-nuclear lysates were resolved by SDS-PAGE using 4%-15% BisTris NUPAGE gels (Invitrogen) and proteins were transferred to membranes (Immunoblot PVDF membranes, Bio-Rad). Membranes were stained with primary antibodies against ETV6/Tel (Novus Biologicals, NBP1-80695), ETV3 (Atlas Antibodies, HPA004794), GP96 (Novus Biologicals, clone 9G10), or actin (Millipore, clone C4), followed by HRP-conjugated secondary antibodies (Jackson Immunoresearch). Some membranes were incubated with “Re-blot Plus” buffer (Millipore).

### Experimental Peritonitis

Cx3cr1-Etv6^{Δ} mice and WT (Etv6^{flox/flox}) littermates were treated with 5 mg of tamoxifen (Sigma) resuspended in Corn oil (Sigma) by oral gavage for 3 consecutive days (day 0-2). On day 5, mice received a fourth gavage of tamoxifen and were injected intra-peritonally with 1 mL of 3.8% brewer's thioglycollate medium (Sigma). Mice were analyzed 3 days after thioglycollate injection.

### Experimental Autoimmune Encephalomyelitis

Cx3cr1-Etv6^{Δ} mice and WT (Etv6^{flox/flox}) littermates were treated with 5 mg of tamoxifen (Sigma) resuspended in Corn oil (Sigma) by oral gavage twice a week, starting one week prior to immunization. Mice were immunized subcutaneously in the back with 100 μg myelin oligodendrocyte glycoprotein (MOG)35-55 peptide (sb-PEPTIDE) emulsified in Incomplete Freud's Adjuvant (Invivogen) supplemented with 4 mg/mL desiccated Mycobacterium Tuberculosis (H37RA, Sigma). Mice were injected intra-peritonally with 200 ng of pertussis toxin from Bordetella Pertussis (Calbiochem) at day 0 and 2 after immunization. Mice were examined daily for clinical signs. In agreement with the local ethics committee, mice were scored as follows: 0 healthy; 0.5 tail weakness; 1 limp tail; 1.5 tail paralysis and hindlimb weakness; 2 tail paralysis and limping of one hindlimb; 2.5 tail paralysis and limping of both hindlimbs; 3 paralysis of tail and both hindlimbs; 3.5 paralysis of tail and both hindlimbs, and weakness in forelimbs. Score 3 was predefined as the humane endpoint of the experiment.

### Statistical Analysis

Wilcoxon matched paired test, Mann-Whitney test and unpaired t test were performed using Prism (GraphPad Software). Statistical details for each experiment can be found in the corresponding figure legend. N corresponds to the number of individual donors or the number of individual mice analyzed.

### Results

ETV3 and ETV6 am More Expressed in Human mo-DCs than mo-Macs In Vitro and In Vivo

We hypothesized that transcription factors differentially expressed between mo-DC and mo-Mac could be involved in their differentiation from monocytes. Our transcriptomic analysis of monocyte-derived cells from clinical samples identified ETV3 and ETV6 as potential candidates (Goudot et al., 2017). To compare ETV3 and ETV6 expression in human monocyte-derived cells, we used our transcriptomics data from cells naturally occurring in vivo in peritoneal ascites or generated in vitro from CD14+ monocytes (Goudot et al., 2017). ETV3 and ETV6 were more expressed in mo-DC when compared to mo-Mac (data not shown) both in vivo and in vitro. To address their potential role in monocyte differentiation, we used our previously published in vitro model allowing the simultaneous differentiation of mo-mac and mo-DC (Goudot et al., 2017). In this model, human monocytes cultured for 5 days with M-CSF, IL-4 and TNF-α differentiate into mo-mac (CD16+), mo-DC (CD1a+) or remain undifferentiated (double negative cells). To verify monocyte purity, and in particular the absence of contaminating DC progenitors, we performed single-cell RNA sequencing (scRNA-seq) on the initial population purified from 2 different donors (data not shown). We found two main populations of monocytes displaying high expression of S100A8 (clusters 0 and 1) or MHCII genes (cluster 2) (data not shown) consistent with the “neutrophil-like” and “DC-like” monocyte populations previously reported21. In addition, we identified a small population of FCGR3A+ monocytes (cluster 3, corresponding to CD14+CD16+ intermediate monocytes), and a negligeable proportion (2% each) of contaminating NK cells (cluster 4) and of monocytes with high ISG expression (cluster 5) (data not shown). These results indicate that our culture model does not contain progenitor cells other than monocytes. To validate the differential expression of ETV3 and ETV6 at the protein level, we measured their expression in sorted mo-DC and mo-Mac by Western Blot. Both transcription factors were more expressed in mo-DCs compared to mo-Macs (data not shown). To characterize their expression kinetics during monocyte differentiation, we measured their expression by RTqPCR at different time points. ETV3 and ETV6 mRNA increased during the first hours in culture with a peak at 3 and 12 hours for ETV3 and ETV6, respectively (data not shown). These results show that ETV3 and ETV6 are expressed at an early stage of monocyte differentiation, suggesting they could play a role in their lineage commitment.

ETV3 and ETV6 are Essential for Human mo-DC Differentiation

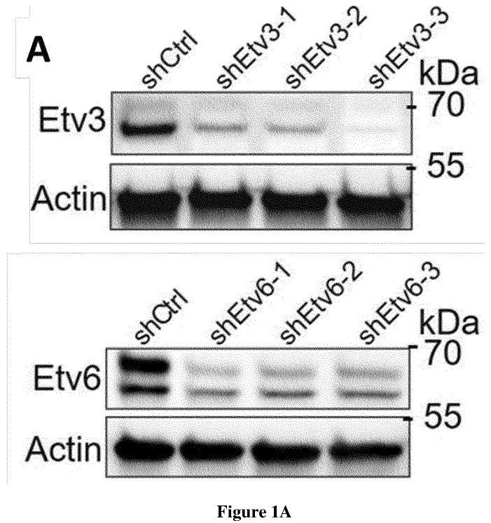

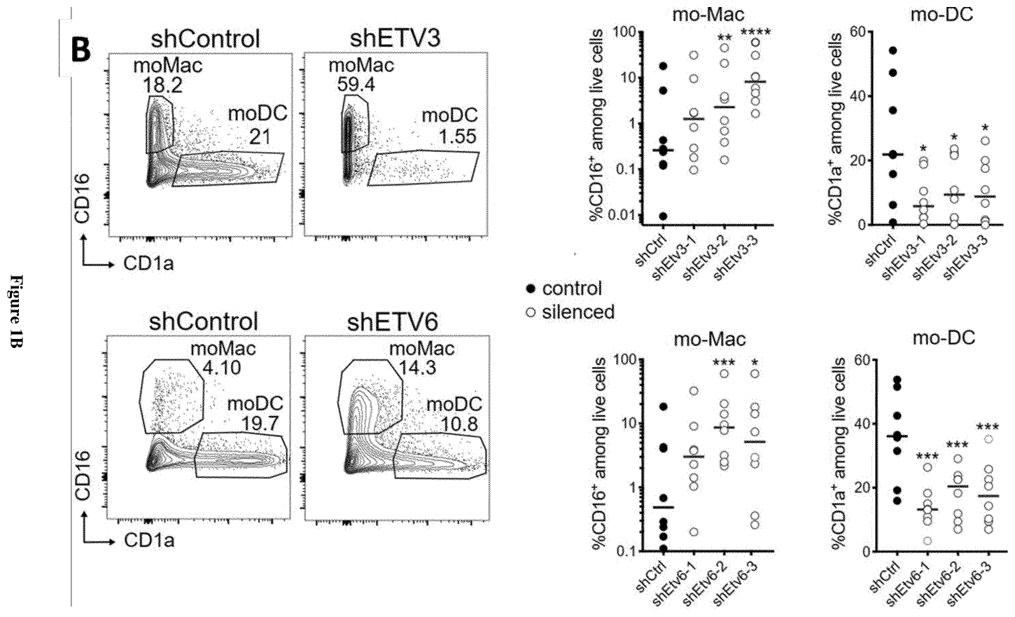

To address the role of ETV3 or ETV6 in monocyte fate commitment, we silenced their expression using a lentivirus expressing a shRNA against ETV3, ETV6 or a scramble sequence. We assessed the effect of silencing on monocyte differentiation after 5 days by staining for phenotypic markers of moDC (CD1a) and moMac (CD16). We used three different shRNA for each molecule, and their efficiency was evaluated by measuring protein expression by Western Blot after 5 days of culture (FIG. 1A). These shRNAs all significantly decreased ETV3 or ETV6 expression with an efficiency between 40-90% (FIG. 1A). Silencing of ETV3 or ETV6 decreased mo-DC and increased mo-Mac differentiation (FIG. 1B). These results show that ETV3 and ETV6 play a key role in mo-DC differentiation. To characterize their expression kinetics during monocyte differentiation, we measured their expression by RTqPCR at different time points. ETV3 and ETV6 mRNA increased during the first hours in culture with a peak at 3 or 6 hours for ETV3 and ETV6, respectively (data not shown). To determine which signals increase their expression, we measured ETV3 and ETV6 mRNA in monocytes upon exposure to M-CSF, in presence or absence of IL-4 and TNF-α (data not shown). ETV3 expression was induced by TNF-α. ETV6 expression was induced by IL-4, with TNF-α sustaining its expression at later time points. These results show that ETV3 and ETV6 are expressed upon exposure to inflammatory signals at an early stage of monocyte differentiation, suggesting they could play a role in their lineage commitment towards mo-DC.

ETV3 and ETV6 Repress mo-Mac Transcriptional Program and Differentiation

ETV3 and ETV6 are transcriptional repressors (Klappacher et al., 2002; Lopez et al., 1999), therefore we hypothesized that they may repress genes involved in mo-Mac differentiation. To decipher the transcriptional network of ETV3 and ETV6, we first investigated the kinetics of their nuclear localization using imaging flow cytometry. To increase the resolution of our analysis, we sought to favor mo-DC differentiation in the culture system by using a modified cytokine cocktail (increased TNFα concentration) (data not shown). We performed intracellular staining of ETV3 or ETV6 after 0, 1, 2, 3 or 6 days of culture. To quantify the expression of ETV3 and ETV6, we gated on ETV3 or ETV6 positive cells (data not shown). The percentage of ETV3+ and ETV6+ cells increased gradually reaching a plateau at day 3 (data not shown). To quantify the nuclear localization of ETV3 or ETV6, we used the ImageStream software to calculate the similarity of the ETV3 or ETV6 channel with the nuclear DAPI staining. High similarity between DAPI and ETV channels (>1.8) indicates a nuclear localization of the transcription factor, while low similarity (<1.8) indicates a cytosolic localization (data not shown). We observed that ETV3 and ETV6 are located in the nucleus until day 3 in around 90% of the cells (data not shown). By contrast, at day 6, ETV3 and ETV6 are located in the cytosol in around 50% of the cells. Because the transcriptional activity of ETV3 and ETV6 requires their nuclear localization, this observation suggests that ETV3 and ETV6 exert their function mainly during the first days of differentiation.

To identify the target genes of ETV3 and ETV6, we performed transcriptomic analysis by bulk RNA-sequencing on monocytes silenced or not for ETV3 or ETV6, at day 3 of differentiation with the modified cytokine cocktail to favor mo-DC development. Then, we performed a differential gene expression analysis using DESeq2 comparing control with silenced samples for ETV3 (data not shown) or ETV6 (data not shown) separately. We defined the differentially expressed genes by a |Log2FC|>0.5 and an adjusted p value <0.05. Comparison of the differentially expressed genes for each shRNA reveals unique transcriptional networks, as most of the genes are specific of ETV3 or ETV6 silencing (data not shown). Using Gene Set Enrichment Analysis (GSEA), we evaluated the enrichment of monocyte, mo-Mac or mo-DC signatures. The mo-Mac signature was enriched in silenced samples, while mo-DC genes were enriched in the control condition (data not shown). These results suggest that ETV3 and ETV6 repress the mo-Mac transcriptional program. To confirm this, we overexpressed ETV6 during monocyte differentiation in conditions where monocytes differentiate exclusively into mo-Macs (culture with M-CSF alone). We validated the forced expression of ETV6 by Western blot after 5 days of culture (data not shown). ETV6 overexpression decreased mo-Mac differentiation (data not shown). Taken together, these results indicate that ETV3 and ETV6 repress mo-Mac differentiation.

### ETV3 and ETV6 Repress Interferon-Stimulated Genes in Human Monocytes

To identify the molecular pathways controlled by ETV3 or ETV6, we performed network analysis. We calculated transcription factor activity using DoRoThEa regulons and VIPER (Garcia-Alonso et al., 2019) (data not shown). STAT1 and STAT2 were the most active transcription factors in silenced samples. We then calculated the enrichment of gene ontology terms (GO, Biological Process) for upregulated genes (data not shown). Type I interferon responses gene sets were enriched in silenced samples. This is consistent with the predicted activity of STAT1 and STAT2, which are known to control the expression of interferon-stimulated genes (ISGs) (Wang et al., 2017). To confirm this, we filtered the differentially expressed genes matrix for known interferon-stimulated genes. Most of the ISGs were more expressed in silenced compared to control samples (data not shown). To determine the in vivo relevance of this finding, we re-analyzed PBMCs single-cell RNA sequencing data from patients carrying a germline mutation of ETV6 (P214L) resulting in loss-of-function (Fisher et al., 2020). We first filtered the data to retain only CD14+ and CD16+ monocytes from healthy and ETV6^{P214L} patients (data not shown). We then interrogated the single-cell data of ETV6^{P214L} and WT monocytes with different gene sets. Genes upregulated upon ETV6 silencing on our in vitro system were enriched in ETV6^{P214L} monocytes compared to WT monocytes (data not shown). Moreover, ETV6^{P214L} monocytes also had a higher enrichment for ISGs than WT monocytes (data not shown), consistent with a previous report (Fisher et al., 2020). These results show that ETV3 and ETV6 repress ISG expression in monocytes in vitro and in vivo in humans. This suggests that ISGs may be involved in the differentiation of monocytes.

Activation of the Type I Interferon Pathway Promotes mo-Mac Differentiation

Given the impact of ETV3 or ETV6 silencing on moDC differentiation, our findings suggest that ISGs may be expressed in our model despite the absence of exogenous interferon in the culture system. To analyze the spontaneous expression of ISG during monocyte differentiation in vitro, we measured MX1, CXCL10 and IFIT3 expression by RTqPCR during the first hours of culture (data not shown). ISG expression peaked at 9 hours. To address whether this phenomenon was specific to our cytokine cocktail, we also cultured monocytes with M-CSF alone, conditions in which monocytes differentiate exclusively into mo-Macs (data not shown). ISG expression was even greater in this setting. To confirm our observations, we quantified by flow cytometry STAT1 phosphorylation, which is required for its transcriptional activity. STAT1 was phosphorylated after 24 hours of culture (data not shown). The percentage of pSTATV⁺ cells was higher with M-CSF than with the three cytokines cocktail, and similar to that induced by exogenous IFNα. However, we could not detect type I interferon secretion in the culture supernatant in any condition. To directly assess the effect of type I interferon stimulation on monocyte differentiation, we cultured monocytes in the presence of IFNα or IFNβ. Type I interferon increased mo-Mac and decreased mo-DC differentiation in a dose-response manner (data not shown). Neither IFNα nor IFNβ affected monocyte-derived cells viability (data not shown). In addition, type I interferon increased the expression of CD163, a macrophage marker, and decreased the expression of CD1b, a DC marker, on the double negative cells (data not shown). Collectively, these results show that activation of the type I interferon pathway promotes mo-Mac differentiation.

ETV3 and ETV6 Control Monocyte Differentiation Independently of their Action on Interferon-Stimulated Genes

To directly test whether ISG expression plays a role in the control of monocyte differentiation by ETV3 or ETV6, we sought to inhibit type I interferon signaling in our culture model using recombinant viral B18R, a soluble receptor of type I interferon that prevents signaling through IFNAR25. Exposure to B18R did not impact the proportions of mo-DC and mo-Mac obtained with or without silencing of ETV3 or ETV6 (data not shown), even though B18R efficiently inhibited ISG expression including MX1, CXCL10, OAS2 and IFIT3 (data not shown). These results indicate that inhibition of the type I interferon pathway does not rescue mo-DC differentiation in the absence of ETV3 or ETV6 expression. We conclude that ETV3 and ETV6 regulate monocyte differentiation independently of their action on ISG.

### Etv6 Represses Interferon-Stimulated Genes In Vivo in Mouse

To validate the physiological relevance of our findings, we employed a mouse model that deletes Etv6 in Cx3cr1-expressing cells after induction with tamoxifen (data not shown). To characterize the cell types targeted by the deletion, we measured a YFP reporter mimicking the endogenous Cx3cr1 expression pattern. YFP was expressed mainly in monocytes and cDCs, and at low levels in pDCs and granulocytes (data not shown). In addition, we measured Etv6 expression by RT-qPCR in cell-sorted populations (data not shown). Etv6 expression was significantly decreased in bone marrow and spleen monocytes of Cx3cr1-Etv6Δ mice, as well as in spleen cDC1 and cDC2 but not pDC. We have previously identified a population of peritoneal mo-DC18. Etv6 was also significantly decreased in peritoneal mo-DC of Cx3cr1-Etv6Δ mice but not in peritoneal mo-Mac or resident macrophages (data not shown). To assess the impact of Etv6 deletion in Cx3cr1-expressing cells on ISGs expression in vivo, we measured by flow cytometry the expression of Sca-1, an interferon-inducible protein (Sisirak et al., 2014). We analyzed immune cells from WT and Cx3cr1-Etv6^{Δ} bone marrow, blood, and spleen. Sca-1 expression was higher in Cx3cr1-Etv6^{Δ} than WT bone marrow monocytes (data not shown). Sca-1 was also more expressed in Cx3cr1-Etv6^{Δ} mice in B cells, T cells and neutrophils in bone marrow, blood, and spleen, and in spleen cDC1, cDC2 and pDC (data not shown). By contrast, deletion of Etv6 in CD11c-expressing cells did not affect Sca-1 expression in spleen and bone marrow B cells (data not shown). These results indicate that the increased ISG expression in Cx3cr1-Etv6^{Δ} mice is due to Etv6 deletion in monocytes. This also suggests that Etv6^{Δ} monocytes spontaneously secrete type I interferon, although we were unable to detect circulating IFNβ (data not shown). To confirm our observations, we analyzed the expression of additional ISGs by RTqPCR in bone marrow monocytes. Isg15, Mx1, Cxcl10 and Ly6a (encoding Sca-1) were more expressed in Cx3cr1-Etv6^{Δ} than in WT monocytes (data not shown) and in peritoneal Etv6^{Δ} mo-DC compared to WT (data not shown). Isg15 and Mx1 were also more expressed in Etv6^{Δ} peritoneal macrophages (data not shown). This widespread spontaneous ISG expression suggests that Etv6 deletion induces type I interferon secretion by Cx3cr1+ cells. Collectively, these results show that Etv6 represses ISG responses in vivo in the steady state.

### Etv6 Controls Mo-DC Differentiation During In Vivo Inflammation in Mouse

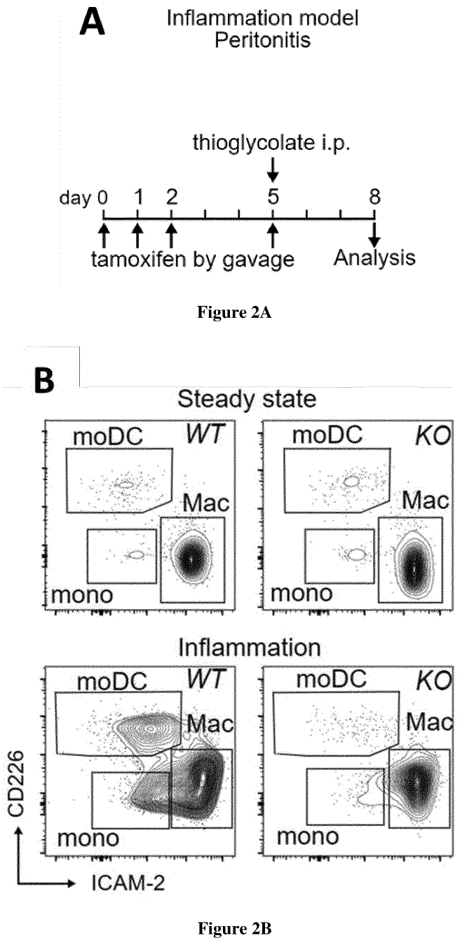

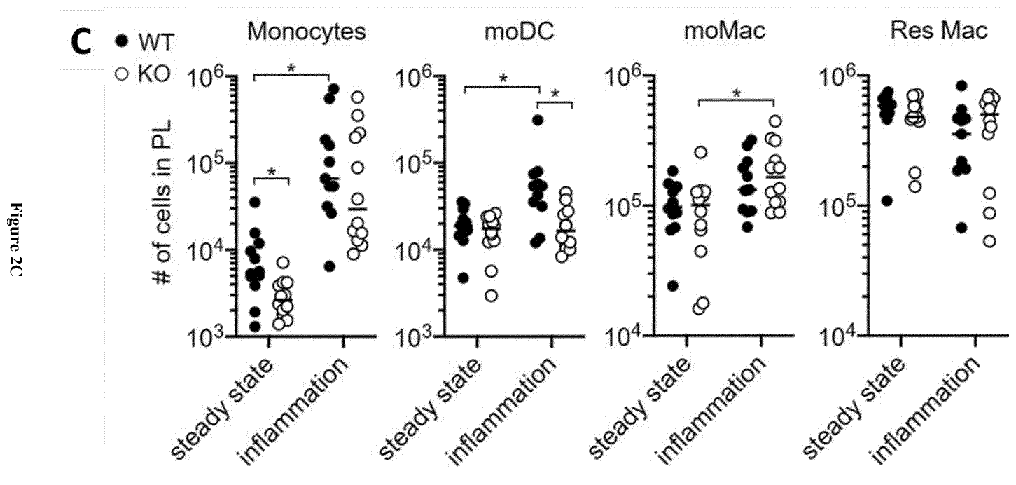

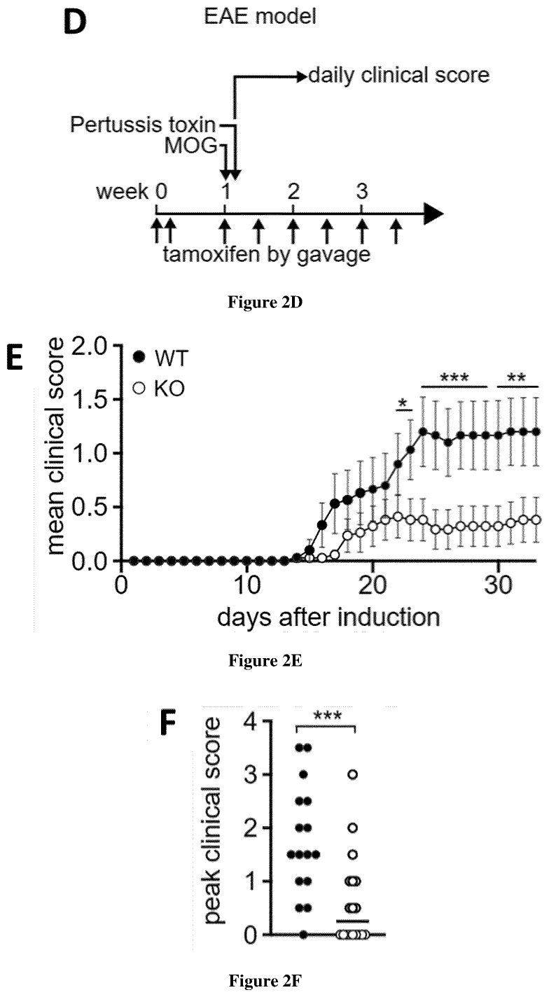

To determine whether Etv6 modulates monocyte differentiation in vivo, we first analyzed monocyte populations in steady-state blood, bone marrow and spleen of Cx3cr1-Etv6^{Δ} mice. The numbers of B cells, T cells, neutrophils, or Ly6C^{high} monocytes were not affected by Etv6 deletion (data not shown) The number of monocyte progenitors was also unchanged (data not shown). The numbers of CD11b⁺CD115⁺Ly6C^{int} and CD11b⁺CD115⁺Ly6C^{neg} monocytes decreased in Cx3cr1-Etv6^{Δ} mice compared to WT. Moreover, the number of spleen cDC2s (both Esam⁻ and Esam⁺) decreased in Cx3cr1-Etv6^{Δ} mice (data not shown). We have previously identified a population of peritoneal mo-DC (Goudot et al., 2017). To address the role of Etv6 in monocyte differentiation in vivo, we analyzed the peritoneal compartment in steady-state and during inflammation (FIG. 2A). In Cx3cr1-Etv6^{Δ} mice, mo-DC and mo-Mac in the steady-state peritoneum were unaffected (FIG. 2B). By contrast, during thioglycolate-induced peritonitis, numbers of mo-DC increased only in WT mice, while mo-Mac increased in Cx3cr1-Etv6^{Δ} mice (FIG. 2C). Monocyte recruitment to the inflamed peritoneum was not different between WT and Cx3cr1-Etv6^{Δ} mice (FIG. 2C), suggesting that the mo-DC/mo-Mac balance was skewed in Cx3cr1-Etv6^{Δ} mice. To confirm that this phenomenon was a monocyte-intrinsic effect, we performed adoptive transfer of CD45.2+WT or Etv6Δ monocytes into the inflamed peritoneum of CD45.1+ recipient mice (data not shown). Transferred monocytes differentiated in situ into mo-DC and mo-Mac (data not shown), however the mo-DC output was significantly decreased in the progeny of Etv6Δ monocytes compared to WT (data not shown). These results show that Etv6^{Δ} monocytes are impaired in their differentiation into moDC during inflammation in mouse, as observed in human monocytes (data not shown).

Finally, we sought to apply our findings to a physio-pathological setting. Mo-DCs have a deleterious role in EAE (Croxford et al., 2015), an animal model for multiple sclerosis (MS). In addition, IFNβ treatment improves disease symptoms and was reported to act primarily on myeloid cells (Prinz et al., 2008). Therefore, we hypothesized that Etv6 deletion in monocytes would ameliorate EAE outcome. We induced EAE in WT and Cx3cr1-Etv6^{Δ} mice by injection of Myelin Oligodendrocyte Glycoprotein (MOG) (FIG. 2D). Cx3cr1-Etv6^{Δ} mice showed less severe symptoms during the course of EAE (FIG. 2E) and reduced incidence (FIG. 2F). These results confirm that Etv6 deletion in monocytes confers protection against severe EAE symptoms. To understand the cellular mechanisms involved in ameliorated EAE outcome, we first analyzed DC populations in the lymph nodes draining the site of MOG injection during induction phase (7 days post-immunization) (data not shown). We found that mo-DC were significantly decreased in the lymph nodes of Cx3cr1-Etv6Δ mice, but not monocytes, neutrophils or other DC subsets (data not shown). MHC II molecules expression by mo-DC was unaffected by Etv6 deletion (data not shown). We hypothesized that decreased mo-DC numbers in Cx3cr1-Etv6Δ mice would reduce the induction of pathogenic CD4+ T cells. To test this, we assessed the presence in lymph nodes of MOG-specific CD4+ T cells using tetramer staining (data not shown). We found that MOG-specific CD4+ T cells were significantly decreased in Cx3cr1-Etv6Δ mice compared to WT, which can explain reduced EAE symptoms in the central nervous system.

Collectively, these results confirm that Etv6 controls monocyte differentiation in vivo in mice during inflammation. We also identify Etv6 in monocytes as a therapeutic target for chronic inflammatory disorders such as MS.

### Discussion:

In this work, we identified ETV3 and ETV6 as molecular regulators of the early stages of monocyte differentiation. We found that ETV3 and ETV6 act as repressors of ISG signaling and of mo-Mac fate commitment. We validated these observations in vivo, showing that mice deficient for Etv6 in monocytes display elevated type I interferon responses and impaired mo-DC differentiation during inflammation. In addition, we found that Etv6 deletion in monocytes reduces the severity of EAE symptoms. Our findings allow a better understanding of the molecular control of monocyte fate decision and identify ETV6 in monocytes as a therapeutic target in inflammatory disorders.

We show that ETV3 and ETV6 repress ISG during monocyte differentiation, and that ETV6 deletion in monocytes induces exacerbated ISG expression in vivo in mouse. This is consistent with previous reports showing that ETV6 is involved in ISG repression in human PBMC (Fisher et al., 2020) and binds to an IFN-stimulated response element in a reporter assay (Kuwata et al., 2002). We also found that genes targeted by ETV3 versus ETV6 were only partially overlapping. This is in line with the observation that ETV7, another member of the ETS transcription repressor family, represses a subset of ISGs, but not all ISGs, in virus-exposed cells (Froggatt et al., 2021). These observations suggest the existence of a specific pattern of target ISGs for each member of the ETV family.

We find that activation of the type I interferon pathway promotes mo-Mac differentiation in our culture system, where human monocytes are exposed to M-CSF, IL-4 and TNF-α. This is consistent with the finding that monocytes differentiated with GM-CSF and IL-4 in the presence of IFN-β display altered phenotype and functional features suggesting impaired mo-DC differentiation, although the re-orientation of their fate was not investigated (Zang et al., 2004). By contrast, it has been reported that exposure to GM-CSF and IFN-α can induce the rapid differentiation of human monocytes into cells with typical mo-DC features, but displaying increased expression of co-stimulatory molecules compared to those obtained using GM-CSF and IL-4 (Blanco et al., 2001; Mohty et al., 2003; Santini et al., 2000). However, whether GM-CSF and IFN-α induce the differentiation of bona fide mo-DC remains unclear, as transcriptomic analysis has revealed a gene signature related to NK cells (Korthals et al., 2007). Our results suggest that STAT1 signaling could dominate over that induced by IL-4, thereby inhibiting mo-DC differentiation in the presence of type I interferon.

We identify ETV3 and ETV6 as key transcriptional regulators of mo-DC differentiation. Additional transcriptional repressors are likely involved in this process, as ETV3 or ETV6 transcriptional activity requires their association with co-repressors. In particular, ETV6 has been shown to associate with IRF8 in a murine macrophage-like cell line (Kuwata et al., 2002), in a human monocyte-like cell line (Huang 2010) and in mouse CD4 T cells (Humblin et al., 2017). While IRF8 is essential for monocyte development from their progenitors (Kurotaki et al., 2013; Sichien et al., 2016), whether it participates in mo-DC or mo-Mac differentiation is unknown. ETV6 has also been reported to associate in human PBMC with NCOR2 (Fisher et al., 2020), which regulates some of the IL4-induced genes during human mo-DC differentiation (Sander et al., 2017). In a human monocyte-like cell line, ETV3 was shown to associate with the repressor DP103, which interacts with the histone deacetylases HDAC2 and HDAC5 (Klappacher et al., 2002). Moreover, ETV6 recruits HDAC3 to the repressor complex in murine cell lines and in human PBMC (Fisher et al., 2020; Kuwata et al., 2002; Wang and Hiebert, 2001). While a specific role for histone deacetylation in mo-DC fate commitment has not been described, it would be consistent with the fact that remodeling of histone acetylation occurs during monocyte differentiation (Nicholas et al., 2015). Further work is needed to unravel the exact mechanism and molecular partners for the repression of ETV3 and ETV6 target genes in monocytes.

Monocyte-derived cells have been shown to play a central role in neuroinflammation. Mice deficient for CCR2 or its ligand, in which monocytes cannot exit the bone marrow, are resistant to EAE or develop milder disease depending on strains (Gaupp et al., 2003; Huang et al., 2001; Izikson et al., 2000; Mildner et al., 2009). In addition, blocking monocyte recruitment using a pharmacological inhibitor diminishes the incidence and severity of EAE (Ge et al., 2012). Monocyte depletion after EAE onset also reduces inflammation and disease symptoms (Getts et al., 2014; Mildner et al., 2009; Moreno et al., 2016). Mo-DC and mo-Mac appear to play different roles during EAE. Mo-DC induce pathogenic Th17 cells by secreting IL-23 (Croxford et al., 2015). By contrast, mo-Mac display specific anti-inflammatory features during the resolution phase of EAE (Giles et al., 2018; Greenhalgh et al., 2016; Locatelli et al., 2018). In MS patients, monocyte recruitment is particularly increased in demyelinated areas (Lagumersindez-Denis et al., 2017). Histological analysis also evidenced the presence around active MS lesions of myeloid cells that have a phenotype consistent with mo-DC and that are found interacting with numerous lymphocytes in situ (Henderson et al., 2009). Specifically blocking monocyte differentiation into mo-DC, while preserving mo-Mac development, could therefore provide clinical benefits in neuroinflammation. Our results identify ETV6 as a candidate target to re-orient monocyte fate decision for therapeutic strategies.

Collectively, our findings suggest that active repression of mo-Mac differentiation is required to allow monocytes to commit to the mo-DC fate, when provided with the appropriate external cues. Given the central role of mo-DC in fueling pathogenic inflammation in numerous chronic inflammatory diseases, our work should have important implications for the therapeutic manipulation of monocyte differentiation.

### REFERENCES

Throughout this application, various references describe the state of the art to which this invention pertains. The disclosures of these references are hereby incorporated by reference into the present disclosure.
Andrews, S., Krueger, F., Seconds-Pichon, A., Biggins, F., and Wingett, S. (2015). FastQC. A quality control tool for high throughput sequence data. Babraham Bioinformatics. Babraham Inst. 1, 1.
Arnold, I. C., Mathisen, S., Schulthess, J., Danne, C., Hegazy, A. N., and Powrie, F. (2016). CD11c+ monocyte/macrophages promote chronic Helicobacter hepaticus-induced intestinal inflammation through the production of IL-23. Mucosal Immunol. 9, 352-363.
Bhatia, M., Landolfi, C., Basta, F., Bovi, G., Ramnath, R. D., de Joannon, A. C., and Guglielmotti, A. (2008). Treatment with bindarit, an inhibitor of MCP-1 synthesis, protects mice against trinitrobenzene sulfonic acid-induced colitis. Inflamm. Res. 57, 464-471.
Blanco, P., Palucka, A. K., Gill, M., Pascual, V., and Banchereau, J. (2001). Induction of Dendritic Cell Differentiation by IFN-α in Systemic Lupus Erythematosus. Science (80-). 294, 1540 LP-1543.
Blighe, K., Rana, S., and Lewis, M. (2018). EnhancedVolcano: Publication-ready volcano plots with enhanced colouring and labeling.
Coillard, A., and Segura, E. (2019). In vivo Differentiation of Human Monocytes. Front. Immunol. 10, 1-7.
Croxford, A. L., Lanzinger, M., Hartmann, F. J., Schreiner, B., Mair, F., Pelczar, P., Clausen, B. E., Jung, S., Greter, M., and Becher, B. (2015). The Cytokine GM-CSF Drives the Inflammatory Signature of CCR2+ Monocytes and Licenses Autoimmunity. Immunity 43, 502-514.
Dobin, A., Davis, C. A., Schlesinger, F., Drenkow, J., Zaleski, C., Jha, S., Batut, P., Chaisson, M., and Gingeras, T. R. (2013). STAR: ultrafast universal RNA-seq aligner. Bioinformatics 29, 15-21.
Evans, H. G., Gullick, N. J., Kelly, S., Pitzalis, C., Lord, G. M., Kirkham, B. W., and Taams, L. S. (2009). In vivo activated monocytes from the site of inflammation in humans specifically promote Th17 responses. Proc. Natl. Acad. Sci. 106, 6232 LP-6237.
Fisher, M. H., Kirkpatrick, G. D., Stevens, B., Jones, C., Callaghan, M., Rajpurkar, M., Fulbright, J., Cooper, M. A., Rowley, J., Porter, C. C., et al. (2020). ETV6 germline mutations cause HDAC3/NCOR2 mislocalization and upregulation of interferon response genes. JCI Insight 5.
Froggatt, H. M., Harding, A. T., Chaparian, R. R., and Heaton, N. S. (2021). ETV7 limits antiviral gene expression and control of influenza viruses. Sci. Signal 14, 1194.
Garcia-Alonso, L., Holland, C. H., Ibrahim, M. M., Turei, D., and Saez-Rodriguez, J. (2019). Benchmark and integration of resources for the estimation of human transcription factor activities. Genome Res. 29, 1363-1375.
Garcia, S., Hartkamp, L. M., Malvar-Femandez, B., van Es, I. E., Lin, H., Wong, J., Long, L., Zanghi, J. A., Rankin, A. L., Masteller, E. L., et al. (2016). Colony-stimulating factor (CSF) 1 receptor blockade reduces inflammation in human and murine models of rheumatoid arthritis. Arthritis Res. Ther. 18, 75.
Gaupp, S., Pitt, D., Kuziel, W. A., Cannella, B., and Raine, C. S. (2003). Experimental autoimmune encephalomyelitis (EAE) in CCR2(−/−) mice: susceptibility in multiple strains. Am. J. Pathol. 162, 139-150.
Ge, S., Shrestha, B., Paul, D., Keating, C., Cone, R., Guglielmotti, A., and Pachter, J. S. (2012). The CCL2 synthesis inhibitor bindarit targets cells of the neurovascular unit, and suppresses experimental autoimmune encephalomyelitis. J. Neuroinflammation 9, 171.
Getts, D. R., Terry, R. L., Getts, M. T., Deffrasnes, C., Müller, M., van Vreden, C., Ashhurst, T. M., Chami, B., McCarthy, D., Wu, H., et al. (2014). Therapeutic Inflammatory Monocyte Modulation Using Immune-Modifying Microparticles. Sci. Transl. Med. 6, 219ra7 LP-219ra7.
Giles, D. A., Washnock-Schmid, J. M., Duncker, P. C., Dahlawi, S., Ponath, G., Pitt, D., and Segal, B. M. (2018). Myeloid cell plasticity in the evolution of central nervous system autoimmunity. Ann. Neurol. 83, 131-141.
Goudot, C., Coillard, A., Villani, A. C., Gueguen, P., Cros, A., Sarkizova, S., Tang-Huau, T. L., Bohec, M., Baulande, S., Hacohen, N., et al. (2017). Aryl Hydrocarbon Receptor Controls Monocyte Differentiation into Dendritic Cells versus Macrophages. Immunity 47, 582-596.e6.
Greenhalgh, A. D., Passos dos Santos, R., Zarruk, J. G., Salmon, C. K., Kroner, A., and David, S. (2016). Arginase-1 is expressed exclusively by infiltrating myeloid cells in CNS injury and disease. Brain. Behav. Immun. 56, 61-67.
Greter, M., Helft, J., Chow, A., Hashimoto, D., Mortha, A., Agudo-Cantero, J., Bogunovic, M., Gautier, E. L., Miller, J., Leboeuf, M., et al. (2012). GM-CSF Controls Nonlymphoid Tissue Dendritic Cell Homeostasis but Is Dispensable for the Differentiation of Inflammatory Dendritic Cells. Immunity 36, 1031-1046.
Guilliams, M., Mildner, A., and Yona, S. (2018). Developmental and Functional Heterogeneity of Monocytes. Immunity 49, 595-613.
Henderson, A. P. D., Barnett, M. H., Parratt, J. D. E., and Prineas, J. W. (2009). Multiple sclerosis: Distribution of inflammatory cells in newly forming lesions. Ann. Neurol. 66, 739-753.
Hock, H., Meade, E., Medeiros, S., Schindler, J. W., Valk, P. J. M., Fujiwara, Y., and Orkin, S. H. (2004). Tel/Etv6 is an essential and selective regulator of adult hematopoietic stem cell survival. Genes Dev. 18, 2336-2341.
Huang, D., Wang, J., Kivisakk, P., Rollins, B. J., and Ransohoff, R. M. (2001). Absence of Monocyte Chemoattractant Protein 1 in Mice Leads to Decreased Local Macrophage Recruitment and Antigen-Specific T Helper Cell Type 1 Immune Response in Experimental Autoimmune Encephalomyelitis. J. Exp. Med. 193, 713-726.
Humblin, E., Thibaudin, M., Chalmin, F., Derangére, V., Limagne, E., Richard, C., Flavell, R. A., Chevrier, S., Ladoire, S., Berger, H., et al. (2017). IRF8-dependent molecular complexes control the Th9 transcriptional program. Nat. Commun. 8.
Izikson, L., Klein, R. S., Charo, I. F., Weiner, H. L., and Luster, A. D. (2000). Resistance to Experimental Autoimmune Encephalomyelitis in Mice Lacking the Cc Chemokine Receptor (Ccr2). J. Exp. Med. 192, 1075-1080.
Jakubzick, C. V, Randolph, G. J., and Henson, P. M. (2017). Monocyte differentiation and antigen-presenting functions. Nat. Rev. Immunol. 2017 176 17, 349-362.
Kamada, N., Hisamatsu, T., Okamoto, S., Chinen, H., Kobayashi, T., Sato, T., Sakuraba, A., Kitazune, M. T., Sugita, A., Koganei, K., et al. (2008). Unique CD14+ intestinal macrophages contribute to the pathogenesis of Crohn disease via IL-23/IFN-γ axis. J. Clin. Invest. 118, 2269.
Klappacher, G. W., Lunyak, V. V, Sykes, D. B., Sawka-Verhelle, D., Sage, J., Brard, G., Ngo, S. D., Gangadharan, D., Jacks, T., Kamps, M. P., et al. (2002). An induced Ets repressor complex regulates growth arrest during terminal macrophage differentiation. Cell 109, 169-180.
Korthals, M., Safaian, N., Kronenwett, R., Maihofer, D., Schott, M., Papewalis, C., Diaz Blanco, E., Winter, M., Czibere, A., Haas, R., et al. (2007). Monocyte derived dendritic cells generated by IFN-α acquire mature dendritic and natural killer cell properties as shown by gene expression analysis. J. Transl. Med. 5, 46.
Kurotaki, D., Osato, N., Nishiyama, A., Yamamoto, M., Ban, T., Sato, H., Nakabayashi, J., Umehara, M., Miyake, N., Matsumoto, N., et al. (2013). Essential role of the IRF8-KLF4 transcription factor cascade in murine monocyte differentiation. Blood 121, 1839-1849.
Kuwata, T., Gongora, C., Kanno, Y., Sakaguchi, K., Tamura, T., Kanno, T., Basrur, V., Martinez, R., Appella, E., Golub, T., et al. (2002). Gamma Interferon Triggers Interaction between ICSBP (IRF-8) and TEL, Recruiting the Histone Deacetylase HDAC3 to the Interferon-Responsive Element. Mol. Cell. Biol. 22, 7439-7448.
Lagumersindez-Denis, N., Wrzos, C., Mack, M., Winkler, A., van der Meer, F., Reinert, M. C., Hollasch, H., Flach, A., Brühl, H., Cullen, E., et al. (2017). Differential contribution of immune effector mechanisms to cortical demyelination in multiple sclerosis. Acta Neuropathol. 134, 15-34.
Lau, C. M., Tiniakou, I., Perez, O. A., Kirkling, M. E., Yap, G. S., Hock, H., and Reizis, B. (2018). Transcription factor Etv6 regulates functional differentiation of cross-presenting classical dendritic cells. J. Exp. Med. 215, 2265-2278.
Leblond, A.-L., Klinkert, K., Martin, K., Turner, E. C., Kumar, A. H., Browne, T., and Caplice, N. M. (2015). Systemic and Cardiac Depletion of M2 Macrophage through CSF-1R Signaling Inhibition Alters Cardiac Function Post Myocardial Infarction. PLoS One 10, e0137515.
Locatelli, G., Theodorou, D., Kendirli, A., Jordio, M. J. C., Staszewski, O., Phulphagar, K., Cantuti-Castelvetri, L., Dagkalis, A., Bessis, A., Simons, M., et al. (2018). Mononuclear phagocytes locally specify and adapt their phenotype in a multiple sclerosis model. Nat. Neurosci. 21, 1196-1208.
Lopez, R. G., Carron, C., Oury, C., Gardellin, P., Bernard, O., and Ghysdael, J. (1999). TEL is a sequence-specific transcriptional repressor. J. Biol. Chem. 274, 30132-30138.
Love, M. I., Huber, W., and Anders, S. (2014). Moderated estimation of fold change and dispersion for RNA-seq data with DESeq2. Genome Biol. 2014 1512 15, 1-21.
Mildner, A., Mack, M., Schmidt, H., Brück, W., Djukic, M., Zabel, M. D., Hille, A., Priller, J., and Prinz, M. (2009). CCR2+Ly-6Chi monocytes are crucial for the effector phase of autoimmunity in the central nervous system. Brain 132, 2487-2500.
Mildner, A., Schonheit, J., Giladi, A., David, E., Lara-Astiaso, D., Lorenzo-Vivas, E., Paul, F., Chappell-Maor, L., Priller, J., Leutz, A., et al. (2017). Genomic Characterization of Murine Monocytes Reveals C/EBP$β$ Transcription Factor Dependence of Ly6C—Cells. Immunity 46, 849-862.e7.
Mohty, M., Vialle-Castellano, A., Nunes, J. A., Isnardon, D., Olive, D., and Gaugler, B. (2003). IFN-α Skews Monocyte Differentiation into Toll-Like Receptor 7-Expressing Dendritic Cells with Potent Functional Activities. J. Immunol. 171, 3385 LP-3393.
Moreno, M. A., Burns, T., Yao, P., Miers, L., Pleasure, D., and Soulika, A. M. (2016). Therapeutic depletion of monocyte-derived cells protects from long-term axonal loss in experimental autoimmune encephalomyelitis. J. Neuroimmunol. 290, 36-46.
Nicholas, D., Tang, H., Zhang, Q., Rudra, J., Xu, F., Langridge, W., and Zhang, K. (2015). Quantitative proteomics reveals a role for epigenetic reprogramming during human monocyte differentiation. Mol. Cell. Proteomics 14, 15-29.
Prinz, M., Schmidt, H., Mildner, A., Knobeloch, K. P., Hanisch, U. K., Raasch, J., Merkler, D., Detje, C., Gutcher, I., Mages, J., et al. (2008). Distinct and Nonredundant In Vivo Functions of IFNAR on Myeloid Cells Limit Autoimmunity in the Central Nervous System. Immunity 28, 675-686.
Sander, J., Schmidt, S. V, Cirovic, B., McGovern, N., Papantonopoulou, O., Hardt, A. L., Aschenbrenner, A. C., Kreer, C., Quast, T., Xu, A. M., et al. (2017). Cellular Differentiation of Human Monocytes Is Regulated by Time-Dependent Interleukin-4 Signaling and the Transcriptional Regulator NCOR2. Immunity 47, 1051-1066.e12.
Santini, S. M., Lapenta, C., Logozzi, M., Parlato, S., Spada, M., Di Pucchio, T., and Belardelli, F. (2000). Type I Interferon as a Powerful Adjuvant for Monocyte-Derived Dendritic Cell Development and Activity in Vitro and in Hu-Pbl-Scid Mice. J. Exp. Med. 191, 1777-1788.
Segawa, M., Fukada, S., Yamamoto, Y., Yahagi, H., Kanematsu, M., Sato, M., Ito, T., Uezumi, A., Hayashi, S., Miyagoe-Suzuki, Y., et al. (2008). Suppression of macrophage functions impairs skeletal muscle regeneration with severe fibrosis. Exp. Cell Res. 314, 3232-3244.
Segura, E., Touzot, M., Bohineust, A., Cappuccio, A., Chiocchia, G., Hosmalin, A., Dalod, M., Sounelis, V., and Amigorena, S. (2013). Human Inflammatory Dendritic Cells Induce Th17 Cell Differentiation. Immunity 38, 336-348.
Sichien, D., Scott, C. L., Martens, L., Vanderkerken, M., Van Gassen, S., Plantinga, M., Joeris, T., De Prijck, S., Vanhoutte, L., Vanheerswynghels, M., et al. (2016). IRF8 Transcription Factor Controls Survival and Function of Terminally Differentiated Conventional and Plasmacytoid Dendritic Cells, Respectively. Immunity 45, 626-640.
Sisirak, V., Ganguly, D., Lewis, K. L., Couillault, C., Tanaka, L., Bolland, S., D'Agati, V., Elkon, K. B., and Reizis, B. (2014). Genetic evidence for the role of plasmacytoid dendritic cells in systemic lupus erythematosus. J. Exp. Med. 211, 1969-1976.
Subramanian, A., Tamayo, P., Mootha, V. K, Mukherjee, S., Ebert, B. L., Gillette, M. A., Paulovich, A., Pomeroy, S. L., Golub, T. R., Lander, E. S., et al. (2005). Gene set enrichment analysis: A knowledge-based approach for interpreting genome-wide expression profiles. Proc. Natl. Acad. Sci. 102, 15545-15550.
Toh, M.-L., Bonnefoy, J.-Y., Accart, N., Cochin, S., Pohle, S., Haegel, H., De Meyer, M., Zemmour, C., Preville, X., Guillen, C., et al. (2014). Bone- and Cartilage-Protective Effects of a Monoclonal Antibody Against Colony-Stimulating Factor 1 Receptor in Experimental Arthritis. Arthritis Rheumatol. 66, 2989-3000.
Wang, L., and Hiebert, S. W. (2001). TEL contacts multiple co-repressors and specifically associates with histone deacetylase-3. Oncogene 20, 3716-3725.
Wang, W., Xu, L., Su, J., Peppelenbosch, M. P., and Pan, Q. (2017). Transcriptional Regulation of Antiviral Interferon-Stimulated Genes. Trends Microbiol. 25, 573-584.
Zaba, L., J, F.-D., NJ, E., MV, A., I, N., KC, P., J, G., JG, K., and MA, L. (2009). Psoriasis is characterized by accumulation of immunostimulatory and Th1/Th17 cell-polarizing myeloid dendritic cells. J. Invest. Dermatol. 129, 79-88.
Zang, Y. C. Q., Skinner, S. M., Robinson, R. R., Li, S., Rivera, V. M., Hutton, G. J., and Zhang, J. Z. (2004). Regulation of differentiation and functional properties of monocytes and monocyte-derived dendritic cells by interferon beta in multiple sclerosis. Mult. Scler. 10, 499-506.
Zigmond, E., Varol, C., Farache, J., Elmaliah, E., Satpathy, A. T., Friedlander, G., Mack, M., Shpigel, N., Boneca, I. G., Murphy, K. M., et al. (2012). Ly6Chi Monocytes in the Inflamed Colon Give Rise to Proinflammatory Effector Cells and Migratory Antigen-Presenting Cells.
Immunity 37, 1076-1090.

1. A method for blocking differentiation of monocytes into dendritic cells in a subject in need thereof comprising administering to the subject a therapeutic effective amount of a ETV6 or ETV3 inhibitor.

2. The method of claim 1 wherein the subject suffers from an inflammatory disease.

3. The method of claim 2 wherein the subject suffers from peritonitis or multiple sclerosis.

4. The method of claim 1 wherein the ETV6 or ETV3 inhibitor is a siRNA, an antisense oligonucleotide or a ribozyme that blocks the translation of ETV6 or ETV3 mRNA and thus prevents protein translation or increasing mRNA degradation.


SEQUENCE LISTING (2 sequences)

SEQ ID NO: 1 - protein, 512 aa, Homo sapiens
MKAGCSIVEKPEGGGGYQFPDWAYKTESSPGSRQIQLWHFILELLQKEEFRHVIAWQQGEYGEFVIKDPDEVARLWGRRKCKPQMNYDKLSRALRYYYNKRILHKTKGKRFTYKFNFNKLVMPNYPFINIRSSGVVPQSAPPVPTASSRFHFPPLDTHSPTNDVQPGRFSASSLTASGQESSNGTDRKTELSELEDGSAADWRRGVDPVSSRNAIGGGGIGHQKRKPDIMLPLFARPGMYPDPHSPFAVSPIPGRGGVLNVPISPALSLTPTIFSYSPSPGLSPFTSSSCFSFNPEEMKHYLHSQACSVFNYHLSPRTFPRYPGLMVPPLQCQMHPEESTQFSIKLQPPPVGRKNRERVESSEESAPVTTPTMASIPPRIKVEPASEKDPESLRQSAREKEEHTQEEGTVPSRTIEEEKGTIFARPAAPPIWPSVPISTPSGEPLEVTEDSEDRPGKEPSAPEKKEDALMPPKLRLKRRWNDDPEARELSKSGKFLWNGSGPQGLATAAADA

SEQ ID NO: 2 - protein, 452 aa, Homo sapiens
MSETPAQCSIKQERISYTPPESPVPSYASSTPLHVPVPRALRMEEDSIRLPAHLRLQPIYWSRDDVAQWLKWAENEFSLRPIDSNTFEMNGKALLLLTKEDFRYRSPHSGDVLYELLQHILKQRKPRILFSPFFHPGNSIHTQPEVILHQNHEEDNCVQRTPRPSVDNVHHNPPTIELLHRSRSPITTNHRPSPDPEQRPLRSPLDNMIRRLSPAERAQGPRPHQENNHQESYPLSVSPMENNHCPASSESHPKPSSPRQESTRVIQLMPSPIMHPLILNPRHSVDFKQSRLSEDGLHREGKPINLSHREDLAYMNHIMVSVSPPEEHAMPIGRIADCRLLWDYVYQLLSDSRYENFIRWEDKESKIFRIVDPNGLARLWGNHKNRTNMTYEKMSRALRHYYKLNIIRKEPGQRLLFRFMKTPDEIMSGRTDRLEHLESQELDEQIYQEDEC